(19) United States
(12) Patent Application Publication
(10) Pub. No.: US 2025/0009676 A1
(43) Pub. Date: Jan. 9, 2025

(54) COMPOSITIONS AND METHODS FOR LEVODOPA DELIVERY

(71) Applicants: PURDUE PHARMA L.P., Stamford, CT (US); NANOMERICS LTD., London (GB)

(72) Inventors: SAVVAS DIMIOU, London (GB); HAIYONG HUGH HUANG, Princeton Junction, NJ (US); ILONA KUBAJEWSKA, London (GB); RUI J. LOPES, London (GB); ANDREAS G. SCHATZLEIN, London (GB); IJEOMA F. UCHEGBU, London (GB)

(21) Appl. No.: 18/697,878

(22) PCT Filed: Oct. 5, 2022
(86) PCT No.: PCT/US2022/045776

§ 371 Date: Apr. 2, 2024

Related U.S. Application Data
(60) Provisional application No. 63/262,162, filed on Oct. 6, 2021.

(51) Int. Cl. A61K 9/51; A61K 9/00; A61K 31/198; B82Y 5/00

(52) U.S. Cl. CPC A61K 9/5161; A61K 9/0043; A61K 31/198; B82Y 5/00

(57) ABSTRACT

A pharmaceutical composition to treat Parkinson's disease is provided, The pharmaceutical composition is an intranasal formulation containing nanoparticles comprised of a therapeutically effective amount of levodopa or a pharmaceutically acceptable salt thereof. A method of treating Parkinson's disease and/or Parkinson's-like symptoms is also provided. The method includes intranasally administering to a patient in need thereof a nanoparticle formulation comprising a pharmaceutically effective amount of levodopa or a pharmaceutically acceptable salt thereof.

### FIELD OF THE INVENTION

The present invention relates to the field of pharmaceutical compositions for nasal administration and methods of use thereof. Specifically, the present invention relates in certain embodiments to a pharmaceutical composition comprising levodopa (i.e., L-DOPA) or a pharmaceutically acceptable salt thereof for treating nervous system disorders, including Parkinson's disease, through nasal administration.

### BACKGROUND OF THE INVENTION

Parkinson's disease is a progressive nervous system disorder that affects movement. In Parkinson's disease, neurons in the brain gradually become dysfunctional or die. Many of the symptoms are due to a loss of neurons that produce dopamine, a chemical messenger in the brain. When dopamine levels decrease, it causes abnormal brain activity, leading to impaired movement and other symptoms of Parkinson's disease. Symptoms start gradually, sometimes starting with a barely noticeable tremor in just one hand. Tremors are common, but the disorder also commonly causes stiffness or slowing of movement. Although there is currently no cure for Parkinson's disease, pharmacological treatments may significantly improve the symptoms.

Levodopa was approved to treat Parkinson's disease over 50 years ago, and as of today it remains the primary treatment. Levodopa is in a class of medications referred to as dopaminergic anti-parkinsonism agents and works by being converted to dopamine in the brain. It is always co-administered with a decarboxylase inhibitor, such as carbidopa, to limit the proportion of the dose converted to dopamine outside of the brain by preventing the levodopa from being broken down before it reaches the brain. When taken orally, the absorption of levodopa occurs in the upper small intestine, and levodopa's pharmacological activity is impaired by unfavorable absorption kinetics.

When levodopa is used in the treatment of Parkinson's disease, it generates the active drug dopamine in the brain. However, levodopa exhibits low oral bioavailability, limited brain uptake, and peripheral dopamine-mediated side effects, such as nausea, tremor and stiffness. Because of its poor brain bioavailability upon oral administration, this can lead to long-term complications, such as motor fluctuations and dyskinesias.

There exists a need in the art for a method of treating Parkinson's disease and a corresponding pharmaceutical composition that avoids the unfavorable absorption kinetics of standard oral therapy of levodopa.

### SUMMARY

It is an object of certain embodiments of the present invention to provide a pharmaceutical composition of therapeutic agents for treating Parkinson's disease.

It is an object of certain embodiments of the present invention to provide a method of treatment for Parkinson's disease through nasal administration.

It is an object of certain embodiments of the present invention to provide a pharmaceutical composition and method of use thereof that avoids the unfavorable absorption kinetics and/or subsequent toxic effects associated with oral levodopa therapy.

It is an object of certain embodiments of the present invention to provide a system comprising a nasal administration device containing the pharmaceutical compositions disclosed herein.

It is an object of certain embodiments of the present invention to provide a method of manufacturing the pharmaceutical compositions and systems disclosed herein.

The above objects and others may be achieved by the present invention which in certain embodiments is directed to a method of treating Parkinson's disease and/or Parkinson's-like symptoms comprising intranasally administering to a patient in need thereof a nanoparticle formulation comprising a pharmaceutically effective amount of levodopa or a pharmaceutically acceptable salt thereof and at least one carbohydrate polymer, wherein the at least one carbohydrate polymer comprises N-palmitoyl-N-monomethyl-N,N-dimethyl-N,N,N-trimethyl-6-O-glycolchitosan (“GCPQ”), quaternary ammonium hexadecyl glycol chitosan (“GCHQ”), quaternary ammonium palmitoyl glycol chitosan (“GCQP”), quaternary ammonium oleyl glycol chitosan (“GCQO”), olely betain glycol chitosan (“GCBO”), palmitoyl betaine glycol chitosan (“GCBP”), or a combination thereof.

In other embodiments, the present invention is directed to an intranasal formulation containing nanoparticles comprised of a pharmaceutically effective amount of levodopa or a pharmaceutically acceptable salt thereof and at least one carbohydrate polymer, wherein the at least one carbohydrate polymer comprises N-palmitoyl-N-monomethyl-N,N-dimethyl-N,N,N-trimethyl-6-O-glycolchitosan (“GCPQ”), quaternary ammonium hexadecyl glycol chitosan (“GCHQ”), quaternary ammonium palmitoyl glycol chitosan (“GCQP”), quaternary ammonium oleyl glycol chitosan (“GCQO”), olely betain glycol chitosan (“GCBO”), palmitoyl betaine glycol chitosan (“GCBP”), or a combination thereof.

### BRIEF DESCRIPTION OF THE DRAWINGS

FIG. 1 is a graph representing the stability of L-DOPA concentration after reconstitution of the lyophilized GCPQ-L-DOPA formulation prepared at the mass ratio of GCPQ:L-DOPA of 10:2 and a drug content of 8 mg/mL L-DOPA:

FIGS. 2a and 2b are SEM images of L-DOPA crystals and GCPQ-L-DOPA nano-in-microparticles, respectively:

FIGS. 3a and 3b are SEM images of spray-dried GCPQ-L-DOPA (mass ratio of GCPQ: L-DOPA=10:2) nano-in-microparticles after 1 month of storage at 4° C. and room temperature conditions, respectively:

FIGS. 4a and 4b is a graph illustrating the pharmacokinetics of GCPQ-L-DOPA and L-DOPA in rats, respectively:

FIG. 5a is a graph illustrating the pharmacokinetics in rat brain tissue following intranasal administration of GCPQ-L-DOPA; and

FIG. 5b is a graph illustrating the dopamine concentration in rat brain tissue following intranasal administration.

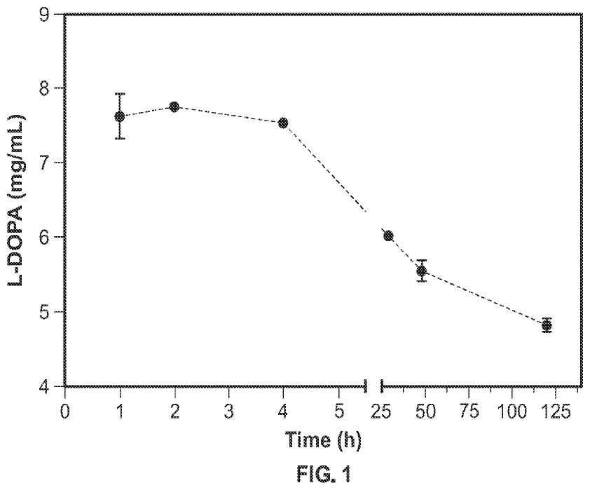

### DETAILED DESCRIPTION

As used herein, the singular forms “a,” “an,” and “the” include plural references unless the context clearly indicates otherwise. Thus, for example, reference to an “excipient” includes a single excipient as well as a mixture of two or more different excipients, and the like.

As used herein, the term “about” in connection with a measured quantity or time, refers to the normal variations in that measured quantity or time, as expected by one of ordinary skill in the art in making the measurement and exercising a level of care commensurate with the objective of measurement. In certain embodiments, the term “about” includes the recited number±10%, such that “about 10” would include from 9 to 11, or “about 1 hour” would include from 54 minutes to 66 minutes.

As used herein, the terms “active agent” and “drug” refer to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates, crystalline forms, metabolites and prodrugs thereof, where the salts, solvates and crystalline forms are pharmaceutically active. In certain embodiments, the “active agent” or “drug” as used herein refers to levodopa.

The term “metabolite” refers to any molecule formed from the metabolism of any of the compounds of the present invention in cells or organismsn (e.g., humans). The term “prodrug” refers to any of the compounds of the present invention which are metabolized in the body to produce a drug.

As used herein, the terms “therapeutically effective amount” and an “effective amount” refer to that amount of an active agent or the rate at which it is administered needed to produce a desired therapeutic result.

The term “patient” means a subject (preferably a human) who has presented a clinical manifestation of a particular symptom or symptoms suggesting the need for treatment, who is treated preventatively or prophylactically for a condition, or who has been diagnosed with a condition to be treated.

The term “subject” is inclusive of the definition of the term “patient” and inclusive of the term “healthy subject” (i.e., an individual, e.g., a human) who is entirely normal in all respects or with respect to a particular condition.

The terms “treatment of” and “treating” include the administration of an active agent(s) with the intent to lessen the severity of to be treated condition.

The terms “prevention of” and “preventing” include the avoidance of the onset of a condition by a prophylactic administration of the active agent(s).

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

The term “concurrently” as used herein means that a dose of one agent is administered prior to the end of the dosing interval of another agent. For example, a dose of nasal levodopa with a particular dosing interval would be concurrently administered with oral levodopa when administered within the dosing interval of the oral levodopa.

The term “simultaneously” as used herein means that a dose of one agent is administered approximately at the same time as another agent, regardless of whether the agents are administered separately via the same or different routes of administration or in a single pharmaceutical composition or dosage form. For example, a dose of nasal levodopa may be administered separately from, but at the same time as, a dose of oral levodopa.

The term “sequentially” as used herein means that a dose of one agent is administered first and thereafter a dose of another agent is administered second. For example, a dose of oral levodopa may be administered first, and thereafter a dose of nasal levodopa may be administered second. The subsequent administration of the second active agent may be inside or outside the dosing interval of the first active agent.

By virtue of certain embodiments of the present invention, there is provided a method of treating Parkinson's disease and/or Parkinson's like symptoms including intranasally administering to a patient in need thereof a nanoparticle formulation comprising a pharmaceutically effectively amount of levodopa or a pharmaceutically salt thereof. Parkinson's like symptoms may include a tremor, which may occur at rest, in the hands or limbs, or may be postural. Parkinson's like symptoms may also be muscular, such as stiff muscles, difficulty standing, difficulty walking, difficulty with bodily movements, involuntary movements, muscle rigidity, difficulty with coordination, rhythmic muscle contractions, slow bodily movement or slow shuffling gait. In some instances. Parkinson's like symptoms may include early awakening, nightmares, restless sleep or sleep disturbances. Parkinson's like symptoms may also include fatigue, dizziness, poor balance or restlessness. Parkinson's like symptoms may also be cognitive, such as amnesia, confusion in evening hours, dementia and/or difficulty thinking and/or understanding. Parkinson's like symptoms may also include difficulty speaking, soft speech and/or voice box spasms. Parkinson's like symptoms may also distort the sense of smell or cause a loss of smell. Parkinson's like symptoms may also include dribbling of urine or leaking of urine. Also, Parkinson's like symptoms may include anxiety or apathy, jaw stiffness or reduced facial expression. Additionally. Parkinson's like symptoms may include blank stare, constipation, depression, difficulty swallowing, drooling, falling, fear of falling, loss in contrast sensitivity, neck tightness, small handwriting, trembling, unintentional writhing and/or weight loss.

In other embodiments, the present invention is directed to an intranasal formulation containing nanoparticles comprised of a pharmaceutically effective amount of levodopa or a pharmaceutically acceptable salt thereof and at least one carbohydrate polymer, wherein the at least one carbohydrate polymer comprises N-palmitoyl-N-monomethyl-N,N-dimethyl-N,N,N-trimethyl-6-O-glycolchitosan (“GCPQ”), quaternary ammonium hexadecyl glycol chitosan (“GCHQ”), quaternary ammonium palmitoyl glycol chitosan (“GCQP”), quaternary ammonium oleyl glycol chitosan (“GCQO”), olely betain glycol chitosan (“GCBO”), palmitoyl betaine glycol chitosan (“GCBP”), or a combination thereof.

In some embodiments, a nanoparticle formulation includes the at least one carbohydrate polymer (e.g., GCPQ) and L-DOPA in a mass ratio of about 2:1 to about 20:1, about 3:1 to about 10:1 or about 10:2 with a drug content of about 1 mg/ml to about 15 mg/mL, about 5 mg/mL to about 10 mg/mL or about 7.5 mg/mL. In some embodiments, a nanoparticle formulation as disclosed herein is stable for up to 4 hours at room temperature after reconstitution and retains a drug loading of 90% or greater, about 92% or greater or about 95% or greater.

In some embodiments, a nanoparticle formulation includes a mixture of different size nanoparticles, e.g., nanoparticles of less than 100 nm and greater than 200 nm. In certain embodiments, the particles are about 20-800 nm in size.

In certain embodiments, the nanoparticles have a positive surface charge of about 20 mv to about 60 mV, about 30 mV to about 60 mV or about 40.5 mV.

In certain embodiments, the nanoparticles are lyophilized. In such embodiments, the size may not be affected after lyophilization.

In some embodiments, the nanoparticle formulation further comprises a vehicle that is acceptable or suitable for intranasal administration. The vehicle that is acceptable or suitable for intranasal administration may be an aqueous solution, a suspension, an ointment, a cream, or a gel. In certain embodiments, the vehicle does not include a thermoreversible gel.

In certain embodiments, the nanoparticle formulation comprises an additional therapeutically active agent.

In some embodiments, the levodopa or pharmaceutically acceptable salt thereof is dissolved or suspended in the vehicle.

In some embodiments, the nanoparticle formulation does not comprise a decarboxylase inhibitor.

In certain embodiments, the pH value of the nanoparticle formulation is from about 4 to about 9, from about 6.5 to about 8.5, from about 7 to about 8 or about 7.5.

In certain embodiments, the levodopa or pharmaceutically acceptable salt thereof is present in the nanoparticle formulation at a concentration of greater than 4 mg/mL, greater than about 6 mg/mL, from about 6 mg/mL to about 10 mg/mL, from about 7 mg/mL to about 9 mg/mL, or about 8 mg/mL.

In certain embodiments, the nanoparticle formulation contacts the olfactory nerves of the patient during administration.

In certain embodiments, the compositions and methods disclosed herein provide a therapeutically effective amount of levodopa or an active metabolite thereof to the brain of the patient.

In some embodiments, the compositions and methods disclosed herein provide a therapeutically effective amount of dopamine to the brain of the patient.

In some embodiments, the method comprises administering the nanoparticle formulation through a nasal device, said nasal device is suitable for delivery of the formulation to the olfactory region of the nose of the patient.

In certain embodiments, the present invention is directed to a system comprising a nasal device is suitable for delivery of a formulation to the olfactory region of the nose of a patient that contains a nanoparticle formulation as disclosed herein.

In certain embodiments, nanoparticles of the nanoparticle formulation are stable.

In certain embodiments, the nanoparticle formulation is lyophilized. The lyophilized formulation may be formulated into the nasal formulations disclosed herein, manufactured, reconstituted just prior to use, or administered as a dry powder.

In some embodiments, the nanoparticles have a mean diameter from about 10 nm to about 800 nm.

In some embodiments, the at least one carbohydrate polymer and the levodopa or pharmaceutically acceptable salt thereof is at a weight ratio from about 1:1 to about 20:1

In certain embodiments, the at least one carbohydrate polymer and the levodopa or pharmaceutically acceptable salt thereof is at a weight ratio of about 10:2.

In certain embodiments, the present invention is directed to an intranasal formulation comprising a nano-in-microparticles composition comprising a pharmaceutically effective amount of levodopa or a pharmaceutically acceptable salt thereof and methods of treating Parkinson's disease thereof.

In certain embodiments, the nano-in-microparticles formulation further comprises at least one carbohydrate polymer with hydrophobic and hydrophilic side groups. In such embodiments, at least one carbohydrate polymer is selected from polysaccharides substituted with hydrophobic and hydrophilic side groups.

In certain embodiments, at least one carbohydrate polymer of the nano-in-microparticles is selected from N-palmitoyl-N-monomethyl-N,N-dimethyl-N,N,N-trimethyl-6-O-glycolchitosan (“GCPQ”), quaternary ammonium hexadecyl glycol chitosan (“GCHQ”), quaternary ammonium palmitoyl glycol chitosan (“GCQP”), quaternary ammonium oleyl glycol chitosan (“GCQO”), olely betain glycol chitosan (“GCBO”), palmitoyl betaine glycol chitosan (“GCBP”), or a combination thereof. In a particular embodiment, the at least one carbohydrate polymer comprises GCPQ.

In some embodiments, a nano-in-microparticles formulation includes the at least one carbohydrate polymer (e.g., GCPQ) and L-DOPA in a mass ratio of about 2:1 to about 20:1, about 3:1 to about 10:1 or about 10:2 with a drug content of about 1% w/w to about 80% w/w; about 10% w/w to about 50% w/w or about 17% w/w. In some embodiments, a nano-in microparticle formulation is stable as a dry powder for up to 30 days at room temperature or stable for up to 4 hours at room temperature after reconstitution in water and retains a drug loading of 90% or greater, about 92% or greater or about 95% or greater when compared to the formulation at the start of the stability study.

In some embodiments, a nano-in-microparticles formulation includes a mixture of different size nanoparticles, e.g., nanoparticles of less than 100 nm and greater than 200 nm. In certain embodiments, the particles are about 20-800 nm in size.

In certain embodiments, the nano-in-microparticles have a positive surface charge of about 20 mv to about 60 mV, about 30 mV to about 60 mV or about 40.5 mV.

In certain embodiments, the nano-in-microparticles are lyophilized. In such embodiments, the size may not be affected after lyophilization.

In some embodiments, the nano-in-microparticles formulation further comprises a vehicle that is acceptable for intranasal administration. The vehicle that is acceptable for intranasal administration may be an aqueous solution, a suspension, an ointment, a cream, or a gel. In certain embodiments, the vehicle does not include a thermoreversible gel.

In certain embodiments, the nano-in-microparticles formulation comprises an additional therapeutically active agent.

In some embodiments, the nano-in-microparticles comprising levodopa or pharmaceutically acceptable salt thereof is dissolved or suspended in the vehicle.

In some embodiments, the nano-in-microparticles formulation does not comprise a decarboxylase inhibitor.

In certain embodiments, the pH value of the nano-in-microparticles formulation is from about 4 to about 9, from about 6.5 to about 8.5, from about 7 to about 8 or about 7.5.

In certain embodiments, the levodopa or pharmaceutically acceptable salt thereof is present in the nano-in-microparticles formulation at a concentration of 1% w/w to about 80% w/w; about 10% w/w to about 50% w/w or about 17% w/w.

In certain embodiments, the nano-in-microparticles formulation contacts the olfactory nerves of the patient during administration.

In some embodiments, the method comprises administering the nano-in-microparticles formulation through a nasal device, said nasal device is suitable for delivery of the formulation to the olfactory region of the nose of the patient.

In certain embodiments, the present invention is directed to a system comprising a nasal device is suitable for delivery of a formulation to the olfactory region of the nose of a patient that contains a nano-in-microparticles formulation as disclosed herein.

In certain embodiments, nanoparticles of the nano-in-microparticles formulation are stable.

In certain embodiments, the nano-in-microparticles formulation is lyophilized. The lyophilized formulation may be formulated into the nasal formulations disclosed herein, manufactured, reconstituted just prior to use, or administered as a dry powder.

In some embodiments, the nano-in-microparticles have a mean diameter from about 1 to about 100 micrometers, about 2-50 micrometers or about 5-30 micrometers

In certain embodiments, the nanoparticles or nano-in-microparticles are spherical, oval, cylindrical, elongated or any other geometry.

In certain embodiments, the intranasal formulation comprises nano-in-microparticles, wherein the nano-in-microparticles have a particle size from about 1 μm to about 30 μm.

In certain embodiments, the intranasal formulation comprises nano-in-microparticles, wherein the nano-in-microparticles are reconstituted having a mixture of small nanoparticles and large nanoparticles, wherein the small nanoparticles are from about 10 nm to about 100 nm or from about 28 nm to about 44 nm and the large nanoparticles are from about 200 nm to about 600 nm from about 300 nm to about 330 nm.

In certain embodiments, the present invention is directed to a pharmaceutical composition comprising a polymeric micellar aggregate, wherein said polymeric micellar aggregates comprise an effective amount of levodopa or a pharmaceutically acceptable salt thereof.

Other embodiments are directed to a method of treating Parkinson's disease comprising intranasally administering to a patient in need thereof a pharmaceutical formulation comprising polymeric micellar aggregates, wherein said polymeric micellar aggregates comprise an effective amount of levodopa or a pharmaceutically acceptable salt thereof.

In certain embodiments, the polymeric micellar aggregates are formed by aggregated individual micelles.

In certain embodiments, the polymeric micellar aggregates have a mean particle size from about 20 nm to about 500 nm and is formed from an amphophilic carbohydrate polymer.

In certain embodiments, the amphophilic carbohydrate polymer is represented by the general formula:

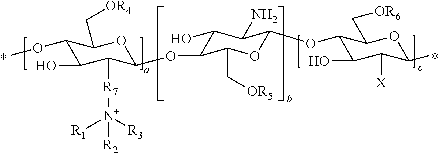

wherein, a+b+c=1 and a is between 0.01 and 0.99, b is between 0 and 0.98 and c is between 0.01 and 0.99,
X is a hydrophobic group;
R₁, R₂ and R₃ are each independently hydrogen or a substituted or unsubstituted alkyl group;
R₄, R₅ and R₆ are each independently hydrogen, a substituted or unsubstituted alkyl group,
a substituted or unsubstituted ether group, or a substituted or unsubstituted alkene group; and
R₇ may be present or absent, and when present, is an unsubstituted or substituted alkyl group, an unsubstituted or substituted amine group or an amide group; or a salt thereof

In certain embodiments, the hydrophobic group is a substituted or unsubstituted group which is an alkyl group, an alkenyl group, an alkynyl group, an aryl group, a polyoxa C₁-C₄ alkylene group or a hydrophobic polymeric substituent.

In certain embodiments, the hydrophobic polymeric substituent is a poly (lactic acid) group, a poly (lactide-co-glycolide) group or a poly (glycolic acid) group.

In certain embodiments, the polymeric micellar aggregates form nanoparticles with the levodopa or the pharmaceutically acceptable salt thereof. Such nanoparticles may have a diameter, e.g., between about 20 nm to about 200 nm.

In certain embodiments, the polymeric micellar aggregates have a minimum mean particle size of at least 100 nm and a maximum mean particle size of 400 nm.

### EXAMPLES

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1

A study was conducted to investigate whether levodopa can be used with a GCPQ nano-in-microparticles. A levodopa dry powder nano-in-microparticulate of Example 1 was prepared as follows. GCPQ (40 mg/mL, 0.5 mL) and levodopa (“L-DOPA”) (3 mg/mL, 1.34 mL) by vigorous mixing for 5 minutes followed by probe sonication (MSE Soniprep 150, MSE London, UK) for 1 minute on ice with the instrument set at an amplitude of 7. GCPQ-L-DOPA nanoparticles were also lyophilized overnight after the addition of the cryoprotectant, sucrose (8.15 mg/mL) at a GCPQ:L-DOPA: sucrose weight ratio of 10:2:7.5.

### Example 2

GCPQ-L-DOPA nanoparticles of Example 2 were prepared from GCPQ (50 mg/mL) and L-DOPA (3 mg/mL) in distilled water (26.67) at a total solid concentration of 1.4% (w/v) and a GCPQ, L-DOPA mass ratio of 10:2. The resulting nanoparticle dispersion was spray-dried to give GCPQ-L-DOPA nano-in-microparticles (Büchi Nano Spray Dryer B290, Büchi Labortechnik AG, Switzerland, inlet temperature=130° C., outlet temperature=65-75° C., aspirator rate=35 m³/hr, spray rate=3 mL/min, gas flow=357 L/hr). The yield was 400 mg and the % yield was 83.3%.

### Characterization of Examples 1 and 2

L-DOPA analysis was performed by high performance liquid chromatography (HPLC). The analysis was performed by UV detection at a fixed wavelength of 280 nm. Separation and quantification were carried out in a Synergi™ Polar-RP analytical column (4 μm particle size, 4.6 mm×250 mm, 80A, Phenomenex, UK) eluted with a mobile phase consisting of water, acetonitrile, trifluoroacetic acid (94.99:5:0.01 (v/v)) at a flow rate of 1 mL/min (column temperature=30° C., injection volume=10 μL, calibration curve for quantification: y=11.676×+1.143, r²=0.999, calibration curve range=1.6-200.0 μg mL⁻¹, n=4 separate experiments, retention time for L-DOPA=3.9 min, total run time=10 min). Data analysis was performed via Agilent Chemstation.

The GCPQ-L-DOPA nano-in-microparticles of Example 2 were dispersed in water and mixed for 30-45 seconds to regenerate nanoparticles to a final concentration of 8 mg/ml of L-DOPA. The L-DOPA content in all formulations were quantified in the supernatant after centrifugation (2,5000 rpm for 2 minutes, Biofuge Fresco, Thermo Scientific, Sweden) to remove any non-encapsulated drug. An aliquot of the supernatant (50 μL) was diluted 20 times with the mobile phase and the solution injected on to the column for analysis. L-DOPA loading was calculated using the following formula:

$$L - DOPA Loading = \frac{( L - DOPA in supernatant )}{Total L - DOPA} \times 100$$

All standards were diluted in the mobile phase prior to analysis by HPLC. The method was validated with respect to linearity, accuracy, precision, limit of quantification (LOQ) and limit of detection (LOD).

### Size and Surface Change in Example 1

The particle size of the nanoparticles of Example 1 was determined using dynamic light scattering (DLS: Zetasizer Nano ZS, Malvern Instruments, UK) at a scattering angle of 173°, a temperature of 25° C., and a wavelength of 633 nm. Samples containing about 8 mg/mL L-DOPA were diluted 10 times with water. The particle size measurements were performed on the diluted samples. Three measurements were performed on each sample and a mean and standard deviation were generated, along with the polydispersity index (PDI). The surface charge of the nanoparticles of Example 1 was also determined with the Zetasizer Nano ZS by loading the diluted samples into zeta potential cuvettes (DTS1070, Malvern Instruments, UK).

### Size Distribution in Example 2

The size distribution of the spray dried nano-in-microparticles of Example 2 was determined by laser scattering using a Malvern Mastersizer 3000 (Malvern Instruments Ltd, Worcestershire, UK). Approximately 10 mg of an aliquot of the powder was applied to the sample feeding tray. Air was used as the dispersion medium for the microparticles from the sample feeding tray to the sample cell. The microparticle size distribution of GCPQ-L-DOPA was characterized by the D₁₀, D₅₀ and D₉₀.

Morphology studies with Scanning Electron Microscopy (SEM)

A strip of double-sided carbon tape was placed on an SEM stub. The nano-in-microparticles of Example 2 were spread across the surface of the tape and compressed air was used to remove loose microparticles. The samples were coated with a 20 nm gold sputter before measurement.

### Stability of GCPQ-L-DOPA Formulations

An aliquot of the GCPQ-L-DOPA nanoparticle formulation of Example 1 was lyophilized. The lyophilized GCPQ-L-DOPA formulations (n=3 separate experiments) were stored in glass vials at room temperature (RT, 16-25° C.), 4° C. and −30° C. for 5 months. Spray-dried GCPQ-L-DOPA formulations of Example 1 (n=3 separate experiments) were stored in glass vials at room temperature (RT, 16-25° C.) and 4° C. for 1 month. At predetermined timepoints spray-dried GCPQ-L-DOPA nano-in-microparticles of Example 2 were characterized for their morphology and microparticle size distribution as described above. Further, lyophilized and spray dried GCPQ-L-DOPA formulations were resuspended to 8 mg/ml of L-DOPA to evaluate L-DOPA loading, nanoparticle size and surface change as described above.

TABLE 1
Stability parameters of lyophilized GCPQ-L-DOPA 10:2 formulations stored
at −30° C., 4° C., and RT conditions for 5 months (n = 3, mean ± SD).

| | L-DOPA | | | | | |
|---|---|---|---|---|---|---|
| | concentration | | | | | |
| | (mg/mL) and | | | | Zeta | |
| Formulation | encapsulation | Z-average | Peak 1 | Peak 2 | Potential | |
| (Time) | efficiency (%) | (nm) | (nm) | (nm) | (mV) | PDI |
| GCPQ-L- | 7.51 ± 0.06 | 140 ± 14 | 289 ± 22 | 19 ± 3 | 40.1 ± 2.5 | 0.670 ± 0.141 |
| DOPA (0 | (95%) | | (84 ± 3) | (12 ± 2) | | |
| month) | | | | | | |
| GCPQ-L- | 7.45 ± 0.32 | 127 ± 59 | 298 ± 44 | 21 ± 8 | 32.1 ± 3.5 | 0.820 ± 0.187 |
| DOPA - | (93%) | | (72 ± 9) | (20 ± 7) | | |
| 30° C. (5 | | | | | | |
| months) | | | | | | |
| GCPQ-L- | 7.17 ± 0.57 | 167 ± 20 | 292 ± 26 | 18 ± 3 | 28.5 ± 4.2 | 0.614 ± 0.152 |
| DOPA 4° C. | (90%) | | (84 ± 3) | (10 ± 2) | | |
| (5 months) | | | | | | |
| GCPQ-L- | 7.15 ± 0.73 | 128 ± 48 | 308 ± 52 | 23 ± 5 | 31.1 ± 5.0 | 0.664 ± 0.252 |
| DOPA RT | (89%) | | (67 ± 10) | (22 ± 9) | | |
| (5 months) | | | | | | |

As shown in Table 1, the GCPQ-L-DOPA formulation of Example 1 was stable when stored lyophilized at different storage conditions. GCPQ was able to maintain L-DOPA stably encapsulated at all storage conditions tested for at least five months, with only a small reduction (2-6%) in L-DOPA loading. Resuspended formulations presented a bimodal particle size distribution with a main peak at 260-280 nm (70-75%) and a secondary peak at 20-30 nm (20-25%) that was maintained for up to 5 months storage. Although particle surface charge had minor reductions throughout the stability study, it remained highly positive (>25 mV) for up to 5 months regardless of the storage conditions.

### GCPQ-L-DOPA Nano-In-Microparticles

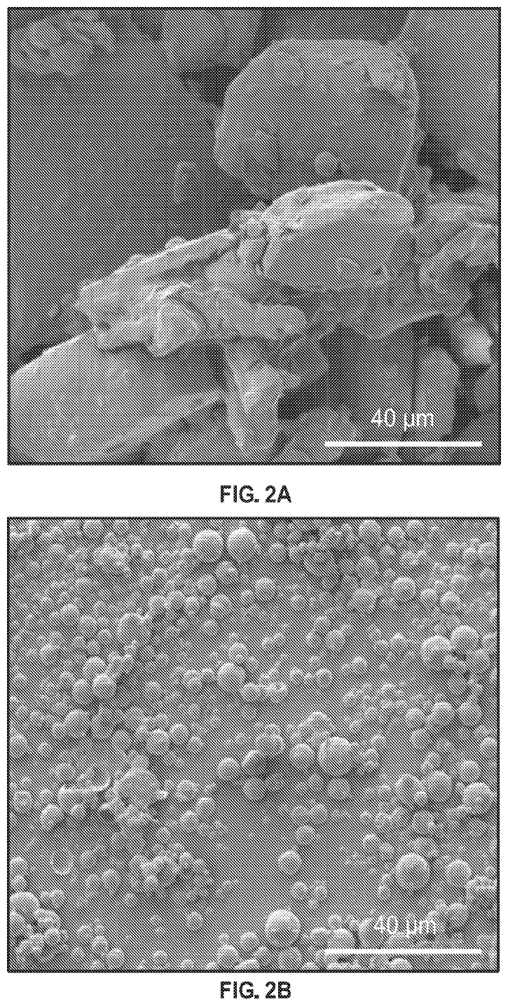

GCPQ-L-DOPA nano-in-microparticles were prepared by spray drying the GCPQ-L-DOPA (mass ratio=10:2) formulation. The spray dryer settings were optimized to increase the yield of the recovered GCPQ-L-DOPA nano-in-microparticles (volume mean diameter=6.15 μm, D₁₀=2.19±0.41, D₅₀=5.27±0.78, D₉₀=11.0±1.48). L-DOPA crystals (as supplied by manufacturer) and GCPQ-L-DOPA nano-in-microparticles were analyzed by SEM, as shown in FIGS. 2a and 2b.

SEM analysis of L-DOPA crystals and GCPQ-L-DOPA suggested that both L-DOPA and GCPQ were thoroughly mixed in the GCPQ-L-DOPA nano-in-microparticles, with no evidence of free L-DOPA as indicated by the absence of L-DOPA crystals in FIG. 2b. Further, GCPQ-L-DOPA nano-in-microparticles had a spherical form, with a non-smooth surface, and a particle size around 1-20 μm, which corresponds well with the particle size determined via the Malvern Mastersizer.

The GCPQ-L-DOPA nano-in-microparticle formulations were also evaluated in terms of drug loading, nanoparticle size, and surface charge after reconstitution of the microparticles in water to achieve a 2 mg/mL L-DOPA concentration. Starting with a GCPQ: L-DOPA mass ratio of 10:2 (17% L-DOPA), a 19.5% L-DOPA content was determined. Assuming no loss of GCPQ during the spray-drying process, this indicated no evidence of L-DOPA degradation during spray drying despite the high temperature (130° C.) used.

TABLE 2
Physicochemical properties of L-DOPA and the GCPQ-
L-DOPA 10:2 formulation before and after spray
drying (n = 3, mean ± SD, ND = not determined).

| | | | | Zeta | |
|---|---|---|---|---|---|
| Formu- | | Peak 1 | Peak 2 | Potential | |
| lation | Z-Average | (nm) | (nm) | (mV) | PDI |
| GCPQ-L- | 76.0 ± 11.0 | 264 ± 36 | 26 ± 4 | ND | 0.91 ± 0.17 |
| DOPA | | (68 ± 6%) | (29 ± 5%) | | |
| before | | | | | |
| spray | | | | | |
| drying | | | | | |
| GCPQ-L- | 116.9 ± 17.4 | 332 ± 30 | 35 ± 14 | 32.6 ± 8.5 | 0.93 ± 0.13 |
| DOPA | | (74 ± 3%) | (18 ± 3%) | | |
| after | | | | | |
| spray | | | | | |
| drying | | | | | |

As shown in Table 2, reconstitution of GCPQ-L-DOPA nano-in-microparticles generated a mixture of small (28-44 nm) and larger (300-330 nm) nanoparticles with a highly positive surface charge of 32.6 mV. This particle size distribution is very similar to the one observed with the lyophilized GCPQ-L-DOPA formulations. Although there is a small increase in the main peak size, this may be due to the absence of sucrose in the spray-dried formulations.

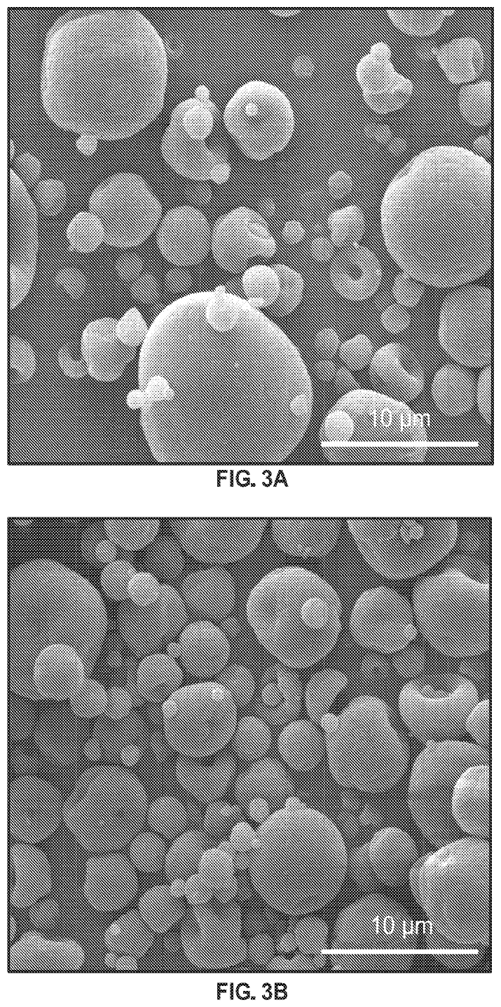

The physical stability of the spray-dried GCPQ-L-DOPA (mass ratio=10:2) formulations was determined over a 1-month storage period at 4° C. and RT by examining key parameters, such as microparticle size, microparticle morphology, drug loading, nanoparticle size, and nanoparticle surface charge. At each timepoint spray-dried formulations were characterized for their microparticle size (Table 3) and morphology (FIG. 3) followed by reconstitution in water to measure the remaining parameters (Table 4).

TABLE 3
Microparticle size distribution stability results of spray-dried GCPQ-
L-DOPA (mass ratio = 10:2) nano-in-microparticles stored at
4° C. and RT conditions for 1 month (n = 3, mean ± SD).

| Formulation | | | |
|---|---|---|---|
| (Time) | D₁₀ (μm) | D₅₀ (μm) | D₉₀ (μm) |
| GCPQ-L- | 2.86 ± 0.20 | 7.16 ± 0.25 | 15.6 ± 0.6 |
| DOPA | | | |
| (0-month) | | | |
| GCPQ-L- | 2.91 ± 0.18 | 7.35 ± 0.30 | 16.0 ± 0.8 |
| DOPA 4° C. | | | |
| (1-month) | | | |
| GCPQ-L- | 2.92 ± 0.11 | 7.26 ± 0.27 | 15.5 ± 0.2 |
| DOPA RT | | | |
| (1-month) | | | |

TABLE 4
Stability results of spray-dried GCPQ-L-DOPA (mass ratio = 10:2) nano-in-
microparticles stored at 4° C. and RT conditions for 1 month (n = 3, mean ± SD).

| | L-DOPA | | | | | |
|---|---|---|---|---|---|---|
| | concentration | | | | | |
| | (mg/mL) and | | | | Zeta | |
| Formulation | encapsulation | Z-average | Peak 1 | Peak 2 | Potential | |
| (Time) | efficiency (%) | (nm) | (nm) | (nm) | (mV) | PDI |
| GCPQ-L- | 7.43 ± 0.18 | 100.7 ± 12.1 | 380.9 ± 21.9 | 40.4 ± 2.7 | 40.1 ± 2.5 | 0.91 ± 0.03 |
| DOPA | (93%) | | (72 ± 1%) | (21 ± 2%) | | |
| (0-month) | | | | | | |
| GCPQ-L- | 7.32 ± 0.18 | 105.6 ± 15.2 | 286.0 ± 6.0 | 40.6 ± 3.0 | 45.6 ± 3.7 | 0.92 ± 0.04 |
| DOPA 4° C. | (92%) | | (67 ± 3%) | (26 ± 2%) | | |
| (1-month) | | | | | | |
| GCPQ-L- | 7.34 ± 0.14 | 113.3 ± 19.9 | 306.5 ± 41.1 | 43.2 ± 12.7 | 46.4 ± 2.5 | 0.89 ± 0.06 |
| DOPA RT | (92%) | | (67 ± 4%) | (26 ± 3%) | | |
| (1-month) | | | | | | |

As shown in Table 4, the morphology and microparticle size of GCPQ-L-DOPA (mass ratio=10:2) nano-in-microparticles remained stable for a month regardless of the storage conditions. GCPQ-L-DOPA microparticles were spherical, with a non-smooth surface. GCPQ was able to maintain L-DOPA stably encapsulated at all storage conditions with no reduction in L-DOPA loading. Resuspended formulations presented a bimodal particle size distribution with a main peak at 286-381 nm (67-72%), a secondary peak at 40-43 nm (21-26%), and a highly positive surface charge (>40 mV) for up to 1-month regardless of the storage conditions.

To date, a nano-in-microparticle formulation of L-DOPA for intranasal delivery has not been reported. Microparticles are able to form a system of continuous drug release and thus protect drugs from enzymatic degradation. Furthermore, microparticles, which are capable of adsorbing moisture, become hydrated after absorbing water from epithelial cells and thus, as cells are reversibly dehydrated, junction opening and drug absorption are promoted. The inventors hypothesize that the intranasal administration of the GCPQ-L-DOPA microparticle formulation could reduce peripheral levels of dopamine and eliminate the draw backs associated with oral L-DOPA administration. Furthermore, based on the mucoadhesive properties of GCPQ, a controlled release system providing significant levels of dopamine in the brain could be achieved.

The target particle size for deposition in the nasal cavity is 10 μm mass-median aerodynamic diameter (MMAD), whereas a more acceptable particle size range is between 4.8 and 23 μm. Although a smaller microparticle size distribution can be achieved with the appropriate adjustment of the spray dryer settings, as conducted for GCPQ-LENK, the recommended particle size could easily be obtained without use of excipients.

### Pharmacokinetics

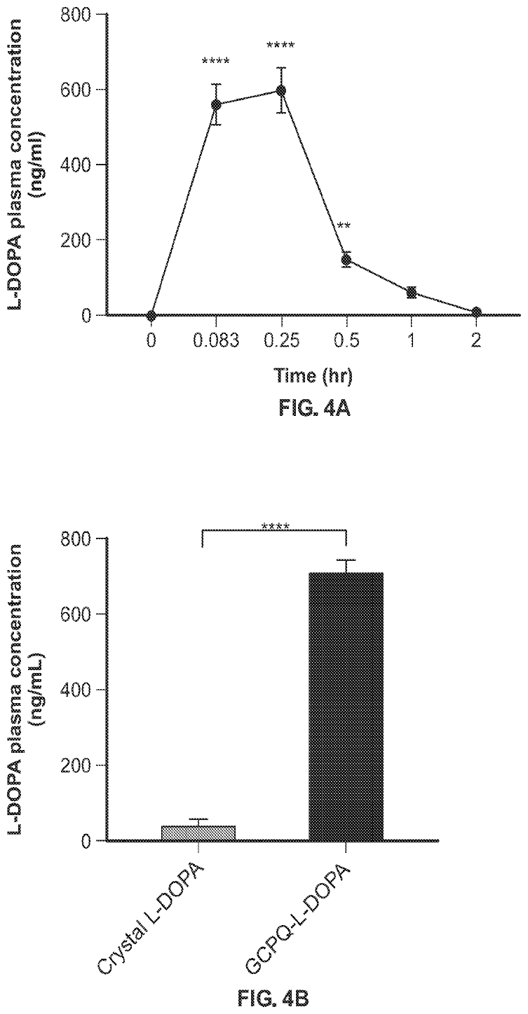

Crystal L-DOPA and GCPQ-L-DOPA formulations at 1.2 mg/kg dose were given intranasally to male Sprague Dawley rats and the levels of L-DOPA and dopamine in plasma and brain were measured. The L-DOPA pharmacokinetics in plasma after the intranasal administration of GCPQ-L-DOPA is shown in FIG. 4a, while the L-DOPA plasma levels at the Tₘₐₓ (0.25 hours) are shown in FIG. 4b.

Following intranasal dosing of crystal L-DOPA, maximum plasma concentrations (53.5±22.7 ng/ml) of L-DOPA were observed 0.25 hours post-dosing. As the plasma level of L-DOPA was below the limit of quantification in 1 out of 4, 1 out of 4, 1 out of 3 and 2 out of 3 animals at the 0.083-, 0.025-, 1- and 2-hour timepoints, respectively, the mean half-life or mean AUC of L-DOPA in plasma could not be determined in these animals. With respect to dopamine, 22 out of 25 samples at various timepoints returned values that were below the limit of quantification with only single animals within a group of four animals at any timepoint recording a value for dopamine of 25-29 ng/ml. The inventors concluded that intranasal L-DOPA alone results in non-therapeutic levels of dopamine in the rat plasma samples.

Following intranasal dosing of GCPQ-L-DOPA, maximum plasma concentrations (mean of 711.8±75.4 ng/mL) of L-DOPA were observed 0.25 hours post-dosing. The mean half-life was 0.427 hours, the AUC_{0-Xhr} of L-DOPA was 343±3.69 hr·ng/mL, and the mean dose-normalized AUCₗₐₛₜ was 286±3.07 hr·kg·ng/(mL mg). Regarding dopamine, the plasma concentrations were below the limit of quantification in all samples apart from one rat (t=0.083 hours. 25.4 ng/mL); therefore, no pharmacokinetic parameters could be determined.

Sato et al reported a basal level of 2.1±0.6 ng/mL for L-DOPA and Nedorubov et al reported a basal level of 1.71±0.63 ng/mL for DA in rat plasma. In this study, the inventors were not able to measure the endogenous plasma concentrations of L-DOPA and dopamine due to the sensitivity of the LC-MS method (limit of quantification=5 ng/mL and 25 ng/ml for L-DOPA and dopamine respectively). Furthermore, rats were fasted overnight which could result in much lower endogenous L-DOPA levels in plasma. Nevertheless, basal L-DOPA plasma levels are negligible when compared with the levels obtained after intranasal administration of GCPQ-L-DOPA in this study.

Interestingly, when L-DOPA is formulated with GCPQ, an approximately 17-fold increase in L-DOPA bioavailability in plasma was observed at Tₘₐₓ compared to the crystal L-DOPA formulation (FIG. 4b). Thus. GCPQ particles appear to transport L-DOPA to the blood more efficiently.

L-DOPA is present in the periphery after intranasal administration in many other studies. However, without a decarboxylase inhibitor, such as carbidopa, only 1% of peripheral L-DOPA can reach the brain as the drug is being rapidly converted to dopamine by the enzyme amino acid decarboxylase. Furthermore, intranasal administration of L-DOPA formulated with GCPQ did not contribute significantly to dopamine plasma levels (values were largely below the limit of quantification). This could be due to the rapid rate of elimination of dopamine and the slow rate of metabolism of L-DOPA in plasma. Therefore, the inventors showed that by not using the oral route, and hence avoiding gastrointestinal wall and hepatic metabolism, they were able to produce substantially lower plasma concentrations of dopamine. Since the debilitating side effects of oral L-DOPA therapy, such as nausea, tremors, and stiffness, are attributed to higher dopamine levels in the peripheral circulation, nasal administration of GCPQ-L-DOPA may minimize these adverse effects by not elevating dopamine concentrations in the blood.

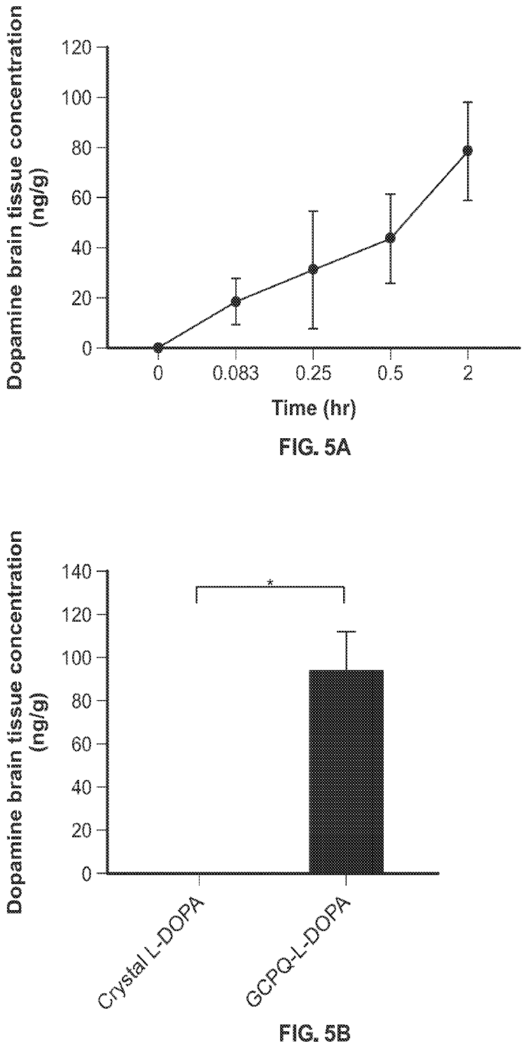

Regarding pharmacokinetics in the brain, all L-DOPA concentrations were below the LOQ (50 ng/ml). Nedorubov et al reported a basal level of 3.4±0.9 ng/g and a Cₘₐₓ of 16.94±1.52 for L-DOPA in rat brain tissue after intranasal L-DOPA administration. Thus, it is possible that the inventors were not able to measure L-DOPA brain levels throughout the study due to the sensitivity of the LC-MS method. Nevertheless, the dopamine basal levels were 96.3 ng/g, which are similar to values obtained in the literature. 99.18±20.5 ng/g. The dopamine pharmacokinetics (dopamine levels in excess of basal levels) in the brain after intranasal administration of GCPQ-L-DOPA and the comparison of the dopamine brain levels following crystal L-DOPA or GCPQ-L-DOPA intranasal administration at Tₘₐₓ (2 hours) are shown in FIG. 5a and FIG. 5b respectively. All brain levels are reported after subtraction of basal levels.

Although L-DOPA levels in the brain were not able to be measured, the inventors obtained an increase in the dopamine brain levels after intranasal administration of GCPQ-L-DOPA ranging between 63-127 ng/g (t=2 hours) above the basal levels. Nedorubov et al reported that dopamine was found in more significant concentrations in rat brains than its precursor. L-DOPA, after intranasal delivery of 3.4 mg/kg L-DOPA compared to 1.2 mg/kg of the present formulated L-DOPA.

Interestingly, the crystal L-DOPA formulation did not result in an increase of dopamine levels in the brain: in fact, they were below the LOQ (FIG. 5b). From this we concluded that the intranasal administration of L-DOPA alone does not yield the pharmacologically relevant levels of entity dopamine. On the other hand, when L-DOPA is formulated with GCPQ, it leads to a statistically significant increase in dopamine concentrations in the brain above the basal levels. The brain dopamine Cₘₐₓ (94.5 ng/g) obtained with the intranasally administered GCPQ-L-DOPA is probably attributed to both nasal cavity-blood circulation-brain pathway as well as the direct nose-to-brain route of L-DOPA. Thus, the delivery routes were not able to be distinguished in this study. There is evidence in the art that show a transport from the nasal mucosa to the olfactory bulb, and the cerebrospinal fluid (CSF). Preferential absorption of intranasally administered compounds into the olfactory bulb or the CSF has been widely reported. Furthermore, the inventors have previously shown direct nose-to-brain delivery using GCPQ nanoparticles. Nevertheless, the nasal cavity is highly vascularized with many blood vessels supplying blood to the nasal cavity. Therefore, the increased L-DOPA levels in the plasma after intranasal administration of GCPQ-L-DOPA (Cₘₐₓ of 711.8 ng/ml) may contribute substantially to the generation of dopamine in the brain by L-DOPA absorption through the nasal cavity-blood circulation-brain route.

Interestingly, dopamine levels kept increasing after 2 hours post-dosing in the study. Without being limiting to a theory, the inventors believe that the mucoadhesive properties of the GCPQ particles may enhance the retention time of L-DOPA in the nares and enable a controlled release system, leading to a stable increase in dopamine levels over the two hour time period (FIG. 5a). Thus, the dopamine increase was attributed to GCPQ-mediated delivery of L-DOPA to the brain via a direct (nose-to-brain) and indirect (nasal cavity-blood circulation-brain) pathway. Although L-DOPA was detected in plasma when administered as L-DOPA alone, it barely converted to dopamine in the brain when not encapsulated in nanoparticles. This demonstrated that nanoparticles facilitate L-DOPA delivery to the brain and effective dopamine production after enzymatic conversion.

From this data, it can be concluded that GCPQ-L-DOPA when administered intranasally results in dopamine targeting the brain with little to no plasma exposure of dopamine. This should improve the tolerability of dopamine. Peripheral conversion of L-DOPA to dopamine appeared to be minimal when L-DOPA was administered intranasally.

In summary, GCPQ was utilized to stably encapsulate L-DOPA into a nano-in-microparticle formulation at a drug content of 19.5% (w/w). The nano-in-microparticle L-DOPA formulation (D₁₀: 2.86 μm, D₅₀: 7.16 μm and D₉₀: 15.6 μm), with accompanying stability data, gives rise to GCPQ-L-DOPA nanoparticles upon reconstitution. Nasal administration of the reconstituted GCPQ-L-DOPA nano-in-microparticles to rats resulted in dopamine concentrations steadily increasing in the brain over time (Cₘₐₓ of 94.5 ng/g. 2 hours post-dosing) with significantly higher levels achieved compared to crystal L-DOPA (below the level of detection, 2 hours post-dosing). This provides evidence of enhanced drug retention in the nares and effective delivery (direct: nose-to-brain and indirect: nasal cavity-blood circulation-brain) through a GCPQ-enabled controlled release system. In addition, nasal administration of GCPQ-L-DOPA resulted in increased L-DOPA availability in plasma (˜17-fold increase compared to crystal L-DOPA 15 minutes post-dose), accompanied by insignificant formation of dopamine in the circulation. The latter suggests that our delivery system could avoid the side effects associated with oral L-DOPA therapy that normally result from increased blood levels of dopamine. Since the brain is the intended site of action of L-DOPA therapy, the brain targeting achieved by the administration of GCPQ-L-DOPA via the nasal route may provide a safer and more efficacious treatment for people with of Parkinson's disease.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A method of treating Parkinson's disease and/or Parkinson's-like symptoms comprising intranasally administering to a patient in need thereof a nanoparticle formulation comprising a pharmaceutically effective amount of levodopa or a pharmaceutically acceptable salt thereof and at least one carbohydrate polymer, wherein the at least one carbohydrate polymer comprises N-palmitoyl-N-monomethyl-N,N-dimethyl-N,N,N-trimethyl-6-O-glycolchitosan (“GCPQ”), quaternary ammonium hexadecyl glycol chitosan (“GCHQ”), quaternary ammonium palmitoyl glycol chitosan (“GCQP”), quaternary ammonium oleyl glycol chitosan (“GCQO”), olely betain glycol chitosan (“GCBO”), palmitoyl betaine glycol chitosan (“GCBP”), or a combination thereof.

2. (canceled)

3. (canceled)

4. (canceled)

5. The method of claim 1, wherein the at least one carbohydrate polymer comprises GCPQ.

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. The method of claim 1, wherein the concentration of levodopa or pharmaceutically acceptable salt thereof in the nanoparticle formulation is from about 6 mg/mL to about 10 mg/mL.

19. (canceled)

20. (canceled)

21. (canceled)

22. The method of claim 1, wherein said method provides a therapeutically effective amount of levodopa to the brain of the patient.

23. The method of claim 1, wherein said method provides a therapeutically effective amount of dopamine to the brain of the patient.

24. (canceled)

25. (canceled)

26. (canceled)

27. (canceled)

28. The method of claim 1, wherein the at least one carbohydrate polymer and the levodopa or pharmaceutically acceptable salt thereof is at a weight ratio from about 1:1 to about 20:1.

29. The method of claim 1, wherein the at least one carbohydrate polymer and the levodopa or pharmaceutically acceptable salt thereof is the weight ratio of about 10:2.

30. An intranasal formulation containing nanoparticles comprised of a pharmaceutically effective amount of levodopa or a pharmaceutically acceptable salt thereof and at least one carbohydrate polymer, wherein the at least one carbohydrate polymer comprises GCPQ, GCHQ, GCQO, GCQP GCBO, or GCBP.

31. (canceled)

32. (canceled)

33. (canceled)

34. (canceled)

35. (canceled)

36. (canceled)

37. The intranasal formulation of claim 30, wherein the intranasal formulation does not comprise a decarboxylase inhibitor.

38. The intranasal formulation of claim 30, wherein the pH value of the intranasal formulation is from about 6 to about 9.

39. (canceled)

40. (canceled)

41. (canceled)

42. (canceled)

43. (canceled)

44. The intranasal formulation of claim 30, wherein the concentration of levodopa or pharmaceutically acceptable salt thereof is from about 6 mg/mL to about 10 mg/mL.

45. (canceled)

46. The intranasal formulation of claim 30, wherein the concentration of levodopa or pharmaceutically acceptable salt thereof is about 8 mg/mL.

47. (canceled)

48. (canceled)

49. (canceled)

50. The intranasal formulation of claim 30, wherein the nanoparticle formulation is lyophilized.

51. (canceled)

52. The intranasal formulation of any claim 30, wherein the at least one carbohydrate polymer and the levodopa or pharmaceutically acceptable salt thereof is at a weight ratio from about 1:1 to about 20:1.

53. The intranasal formulation of claim 52, wherein the at least one carbohydrate polymer and the levodopa or pharmaceutically acceptable salt thereof is at a weight ratio of about 10:2.

54. An intranasal formulation comprising nano-in-microparticles composition comprising a therapeutically effective amount of levodopa or a pharmaceutically acceptable salt thereof.

55. (canceled)

56. (canceled)

57. (canceled)

58. (canceled)

59. The intranasal formulation of claim 54, wherein the nano-in-microparticles formulation is lyophilized.

60. (canceled)

61. (canceled)

62. (canceled)

63. The intranasal formulation of claim 54, wherein the nano-in-microparticles retain at least about 95% drug loading.

64. (canceled)

65. (canceled)

66. (canceled)

67. The intranasal formulation of claim 54, wherein the nano-in-microparticles are reconstituted having a mixture of small nanoparticles and large nanoparticles, wherein the small nanoparticles are from about 28 nm to about 44 nm and the large nanoparticles are from about 300 nm to about 330 nm.

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. A method of delivering levodopa or a pharmaceutically acceptable salt thereof to a patient identified as in need of levodopa therapy, comprising administering to the olfactory region of the nose of the patient an intranasal formulation of claim 30.
